(19) United States
(12) Patent Application Publication
(10) Pub. No.: US 2006/0140870 A1
(43) Pub. Date: Jun. 29, 2006

(54) Method for assessing tumor response to cancer treatment

(75) Inventors: Egidijus Uzgiris, Niskayuna, NY (US); Mohan Amaratunga, Clifton Park, NY (US); Brian Grimmond, Clifton Park, NY (US); Daniel Meyer, Rexford, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/023,138

(22) Filed: Dec. 23, 2004

(51) Int. Cl. A61K 49/14

(52) U.S. Cl. 424/9.36

(57) ABSTRACT

A method for measuring tumor response to a cancer therapy is provided. The method comprises the steps of injecting a polymeric contrast agent in a subject; obtaining a series of magnetic resonance image signals of the contrast agent in the tumor for up to about 60 minutes; and determining a slope of the magnetic resonance image signal as a function of time.

### BACKGROUND

1. Technical Field

The present disclosure relates to a method for assessing tumor response to cancer treatment. More particularly, the present disclosure is related to a magnetic resonance imaging method for determining tumor permeability and fractional blood volume of a tumor.

2. Description of Related Art

Tumor angiogenesis is the recruitment of new blood vessels by a growing tumor from existing neighboring vessels. This recruitment of new microvasculature is a central process in tumor growth and in the potential for aggressive spreading of the tumor through metastasis. All solid tumors require angiogenesis for growth. Thus, the level of angiogenesis is thought to be an important parameter for the staging of tumors. Furthermore, new therapies are being developed which attack the process of angiogenesis for the purpose of attempting to control tumor growth and tumor spread by restricting or eliminating the tumor blood supply. It is therefore of clinical importance to be able to monitor angiogenesis and the affect of therapies in tumors in a noninvasive manner.

To assess tumor growth, two parameters are of primary importance: vascular volume and vascular permeability. Non-invasive methods include a magnetic resonance imaging method with a type of contrast agent that enables measurement of both vascular volume and vascular permeability. For instance, U.S. Pat. No. 6,235,264 involves a magnetic resonance imaging method with a type of contrast agent that enables high sensitivity measurement of both vascular volume and vascular permeability. A magnetic resonance image is taken before and after the introduction of a reptating polymer contrast agent into a subject.

When a substance such as living tissue is subjected to a uniform magnetic field (polarizing field B0), individual magnetic moments of the nuclear spins in the tissue attempt to align with this polarizing filed along the z axis of a Cartesian coordinate system, but precess about the z axis direction in random order at their characteristic Larmor frequency. If the substance, or tissue, is subjected to a magnetic field (excitation field B1) which is in the x-y plane and at a frequency near the Larmor frequency, the net aligned longitudinal magnetization may be rotated, or “tipped”, into the x-y plane to produce a net transverse magnetization. A signal is emitted by the excited spins after the excitation signal BI is terminated. This magnetic resonance imaging (MRI) signal may be received and processed to form an image.

When utilizing MRI signals of this type to produce images, magnetic field gradients, (Gx, Gy, and Gz) are employed. Typically, the region to be imaged is scanned with a series of measurement cycles in which these gradients vary according to the particular localization method being used. The resulting set of received MRI signals is digitized and processed to reconstruct the image using one of many well know reconstruction techniques.

One of the mechanisms employed in MRI to provide contrast in reconstructed images is the T1 relaxation time of the spins. After excitation, a period of time is required for the longitudinal magnetization to fully recover. This period, referred to as the T1 relaxation time, varies in length depending on the particular spin species being imaged. Spin magnetizations with shorter T1 relaxation times appear brighter in MR images acquired using fast, T1 weighted MRI measurement cycles. A number of contrast agents which reduce the T1 relaxation times of neighboring water protons are used as in vivo markers in MR images. The level of signal brightness, i.e. signal enhancement, in T1 weighted images is proportional to the concentration of the agents in the tissue being observed.

Tumor growth as well as tumor volume shrinkage can be measured by MRI. For instance, a current standard method for assessing cancer therapy response is a method which includes measuring tumor volume shrinkage by MRI. Unfortunately, detection of volume response to a cancer treatment requires time duration on the order of a month or several months.

Thus, there remains a need to assess response to therapy in cancer treatment on a time scale of days rather than weeks or months. This need may become even more acute with the advent of anti-angiogenesis drugs and more specific and restrictive chemotherapy agents tailored to a personalized medicine approach.

### SUMMARY

The present invention provides a method for measuring tumor response to a cancer treatment comprising the steps of:

a) injecting a polymeric contrast agent in a subject;

b) obtaining a series of magnetic resonance image signals of the contrast agent in the tumor for up to about 60 minutes; and

c) determining a slope of the magnetic resonance image signal as a function of time

### BRIEF DESCRIPTION OF THE DRAWINGS

While the novel features of the invention are set forth with particularity in the appended claims, the invention, both as to organization and content, will be better understood and appreciated, along with other objects and features thereof, from the following detailed description taken in conjunction with the drawing, in which:

FIG. 1 is a graph of the signal slope determination of a Mat B tumor injected with a Polylysine-Gd-DTPA reptating polymer with a series of images taken at intervals of 3 minutes for the first 10 minutes, then every 5 minutes up to 20 minutes, and finally every 10 minutes up to a final time of 60 minutes.

### DETAILED DESCRIPTION OF THE INVENTION

In a method for assessing the response of a tumor to a cancer treatment, a polymeric contrast agent is intravenously injected in a subject and a series of timed medical images of the tumor sight is obtained via magnetic resonance imaging. The series of timed medical images in conjunction with the polymeric contrast agent enables a straightforward measurement of the permeability of the tumor endothelia through signal changes of the magnetic resonance image as a function of time. Response to a cancer treatment is gauged by measuring the slope of signals of the magnetic resonance image before cancer treatment and after cancer treatment. Additionally, the magnetic resonance image signal at time zero (i.e., the intercept of the signal slope with time) is a measure of the tumor fractional blood volume, which may also change after cancer treatment.

In accordance with the present invention, the polymeric contrast agent is injected into the subject and a series of images at one or more pre-selected tissue sights are taken post injection for a period of up to about 60 minutes. Any combination of intervals for the series of images can be taken as long as a slope can be derived of the signal as a function of time. For example, a series of images can be taken at intervals of 3 minutes for the first 10 minutes, then every 5 minutes up to 20 minutes, and finally every 10 minutes up to a final time of 60 minutes. After the series of images are obtained, the magnetic resonance image signal is plotted as a function to time to determine the slope of the signal change. No rapid sequences in the sub-minute time scale are necessary to derive a signal uptake slope. Tumor permeability can be calculated by using the signal uptake slope and the appropriate parameters. These parameters are: concentration of the polymeric contrast agent in the blood; the R1 longitudinal relaxivity of the polymeric contrast agent; and the T1 of the tumor tissue.

Once the slope has been obtained, the tumor fractional blood volume can be measured. The tumor signal at time zero (i.e., the intercept of the signal slope with time) is a measure of the tumor fractional blood volume, the other hemodynamic parameter of interest.

To determine the response to a cancer treatment, a repeat and comparison of the signal enhancement curve can be taken at a later date post cancer treatment. For instance, a second injection dose of polymeric contrast agent that is identical to the first dose is intravenously injected into the subject and a repeat of the measurement is done at least 24 hours or a few days after the initial measurement. A second slope is obtained and compared to the initial slope. A decrease in the second slope and a negative change of the signal intercept at zero time would indicate a positive response to cancer treatment. Hence, the present invention allows an assessment of the response to a cancer treatment on a time scale of days rather than weeks or months.

The nature of the polymer backbone of the polymeric contrast agent is not critical, provided that the polymer has pendant groups which can be reacted with activated steric hindrance molecules (“SHM”) as described below to provide a polymer having an elongated structure. Suitable pendant groups which may be present in the polymer include, but are not limited to amine groups, carboxyl groups, and hydroxyl groups. Useful polymers include homo- and co- polymers of poly(amino acids), poly(vinyl amine), poly(4-aminostyrene), poly(acrylic acid), poly(methacrylic acid), poly(carboxynorbomene), and dextran. Preferably, the polymer is an amino acid homopolymer or a copolymer of two or more amino acids. Amino acid containing polymers are also known as polypeptides. Preferably, the polypeptide is selected from the group consisting of polylysine, polyglutamic acid, polyaspartic acid, and a copolymer of lysine and either glutamic acid or aspartic acid. More preferably, the polypeptide is polylysine. Other polymers may be used provided that after reaction with the SHM, the resulting polymer has an elongated structure characterized by a molecular length that is in a range between about 5 and about 500 times the cross-sectional diameter of the polymer molecule and a net negative charge in an aqueous environment. In addition, the polymer preferably is of sufficient length to increase the time in which the product circulates in the blood. For polypeptides, the polymer backbone can advantageously be in a range between about 35 and about 1500 amino acid residues long and is preferably in a range between about 100 and about 800 amino acid residues long. The reptating conformation of the polymeric contrast agents in the present invention allows the polymeric contrast agents to translocate across the tumor endothelium with high efficiency. Because the polymeric backbone is synthetic, the length can be tailored to provide desired resistance times in the body. Clearance from the blood is rapid for short molecules, resulting in a short plasma lifetime. Plasma lifetime increases rapidly as the polymers increase in length. For example, where the polymer is a polypeptide, a plateau is reached for a molecular length of about 500 residues and little further change in lifetime occurs. Not only does the use of a synthetic polypeptide provide the ability to modify the polymer length so as to change the blood circulations times to smaller values, but the ability to modify the polymer length to probe small permeability modulations is also provided.

The polypeptide may also be a random copolymer which contains lysine units and either glutamic acid units, aspartic acid units, or both. Glutamic and/or aspartic acid units may constitute from about 20 to about 60 percent of the copolymer. Particularly useful copolymers have glu:lys ratio of about 1:4 to about 6:4. A high content of lysine is believed advantageous for imaging as it allows a high loading of the copolymer with paramagnetic ions. Without wishing to be bound by any theory, it is believed that the presence of glutamic acid residues in the copolymer backbone accomplishes two things. First, it is believed that the glutamic acid residues provide a stiffer initial copolymer backbone for the synthesis of the complete construct. Second, it is believed that the presence of glutamic acid residues in the copolymer promotes extension of the final polymer through charge repulsion. Suitable copolymers can be synthesized using techniques known to those skilled in the art. Suitable copolymers are also commercially available from a variety of sources.

When the polymeric contrast agent contains lysine groups, at least a portion of the lysine groups have a steric hindrance molecule (“SHM”) attached thereto. The SHM is any molecule that by its physical size enforces an elongated conformation by providing steric hindrance between neighboring steric hindrance molecules. Preferably the SHM is neutral in charge or presents negative charges in an aqueous environment along the polymer chain to assist in keeping the polymer backbone straight through coulombic repulsion.

Particularly preferred steric hindrance molecules are molecules that chelate with paramagnetic entities. As those skilled in the art will appreciate, paramagnetic entities include certain transition metals and lanthanide ions. Any molecule known to complex with paramagnetic entities and which is of sufficient size to provide steric hindrance against polymer bending can be used as the SHM. Preferably, the group present on the polymer backbone that is derived from the SHM exhibits a net negative charge in an aqueous environment. Suitable lanthanide ion chelating molecules include, but are not limited to diethylenetriaminepentaacetic acid (DTPA), 1,4,7,10-tetraazacyclododecane-1,4,7,10-tetraacetic acid (DOTA), 1,4,7,10-tetraazacyclododecane-1,4,7,10-tetrakis(2-propionic acid) (DOTMA), 1,4,8,11-tetraazacyclotetradecane-1,4,8,11-tetraacetic acid (TETA), 1,4,7,10-tetraazacyclododecane-1,4,7,10-tetrakis[3-(4-carboxyl)-butanoic acid], 1,4,7,10-tetraazacyclododecane-1,4,7,10-tetrakis(acetic acid-methyl amide), 1,4,7,10-tetraazacyclododecane-1,4,7,10-tetrakis(methylene phosphonic acid), and p-isothiocyanatobenzyl -1,4,7,10-tetraazacyclododecane-1,4,7,10-tetraacetic acid (p-SCN-Bz-DOTA). Preferably, the SHM is 1,4,7,10-tetraazacyclododecane-1,4,7,10-tetraacetic acid (DOTA) or p-isothiocyanatobenzyl-1,4,7,10-tetraazacyclododecane-1,4,7,10-tetraacetic acid (p-SCN-Bz-DOTA). Ligands useful for chelating for other ions (such as, for example, Fe(III), Mn(II), Cu(II), etc.) include bis(thiosemicarbazone) and derivatives, porphyrins and derivatives, 2,3-Bis(2-thioacetamido)propionates and derivatives, N,N′-bis(mercaptoacetyl)-2,3-diaminopropanoate, and bis(aminoethanethiol) and derivatives.

In the present invention, the SHM contains or chelates an image producing entity. Suitable image producing entities include paramagnetic entities and entities which undergo nuclear reaction to emit a particle, such as, for example, an alpha particle, a gamma particle, a beta particle, or a positron. Such imaging entities are known to those skilled in the art. Gamma emitters include, for example, 111 In and 153 Gd. Positron emitters include, for example, 89 Zr, which may be employed in positron emission tomography (PET) imaging. Gadolinium is the preferred paramagnetic entity. Thus, for example, to achieve a MR active agent, a paramagnetic ion can be incorporated into the polymer-SHM product. By way of example, gadolinium can be loaded into chelating DTPA groups by dropwise addition of a gadolinium salt (e.g., GdC13 or gadolinium citrate in 0.1 M HCI (50 mM in Gd)) into a solution (15 mM NaHCO3) containing the polymer-SHM product. The dropwise addition of Gd continues until a slight indication of free Gd (not chelated by available DTPA groups) is noted (small aliquots of polymer solution added to 10 μM of arzenzo IIII in acetate buffer—free Gd turns the dye solution blue). The Gd-loaded highly conjugated polymer is then ready for introduction into a blood vessel of the subject.

Typically, the polymeric contrast agent in accordance with this disclosure is introduced into the subject by injecting the contrast agent intravenously. The dose of the polymeric contrast agent can be in a range between about 0.01 mmoles of chelated imaging metal ion per kilogram of patient body weight and about 0.1 mmoles of chelated imaging metal ion per kilogram of patient body weight.

Typically, to attach the SHM to the polymer backbone, an activating group is provided on the SHM. The activating group present on the SHM can be any group which will react with the polymer. Suitable groups include, but are not limited to mixed carbonate carbonic anhydride groups, amine groups, succinimidyl groups and dicyclohexylcarbodiimide (DCC) groups. Those skilled in the art will readily envision reaction schemes for attaching an activating group to any given SHM.

In particularly preferred methods, a substantially mono-activated steric hindrance molecule (“SHM”) is provided. The term “activated” means that a functional group is provided on the SHM which permits covalent bonding of the molecule to the copolymer chain. By the term “substantially mono-activated” it is meant that about 90% or more of the steric hindrance molecules contain only a single activated site.

In certain embodiments, the conjugated polymer can be used for drug delivery. It is contemplated, for example, that the SHM can itself be a therapeutic agent. It is also contemplated that a therapeutic agent can be attached at a few sites along the substituted polymer chain. By way of example, chemotherapeutic agents (such as, for example, doxorubicin or methotrexate) which have been shown to have activity against tumors can be attached to the conjugated polymer. Even though specific chemotherapy drugs such as doxorubicin and methotrexate are mentioned here, any known chemotherapy drugs capable of being attached to the specific polypeptide being used may be employed. Also, plant and bacterial toxins such as ricin and abrin and the like may be used. For therapy, one could alternatively use a radiotherapeutic agent such as 90Y or 211At.

The therapeutic entity can be attached to the conjugated polymer using techniques known to those skilled in the art. It is also contemplated that therapeutic agents can be used in combination with other types of active agents incorporated into the conjugated polymer. For example, the polymer backbone can be highly conjugated with a non-therapeutic SHM which chelates an image producing entity and a therapeutic agent can appear at only a few sites along the backbone. As another example, the polymer backbone can be highly conjugated with a non-therapeutic SHM, and a therapeutic agent can be bound to the SHM, rather than being bound directly to the polymer backbone.

In other embodiments, the conjugated polymer can be used for targeting specific tissue. It is contemplated, for example, that the SHM can itself be a targeting agent. It is also contemplated that a targeting agent can be attached at a few sites along the substituted polymer chain. The targeting agent can be attached to the conjugated polymer using techniques known to those skilled in the art. It is also contemplated that targeting agents can be used in combination with other types of active agents incorporated into the conjugated polymer. For example, the polymer backbone can be highly conjugated with a non- targeting SHM which chelates an image producing entity and a targeting agent can appear at only a few sites along the backbone. As another example, the polymer backbone can be highly conjugated with a non- targeting SHM, and a targeting agent can be bound to the SHM, rather than being bound directly to the polymer backbone.

The present polymeric contrast agents preferably have a cross sectional diameter which is larger than that of the pores of normal endothelium such that they are contained within the blood vessels in normal tissue but have a cross sectional diameter smaller than that of the pores of the vessels in tumor tissue such that they may readily pass out of the pores and into the interstitial space. Polymeric contrast agents having a diameter in a range between about 20 Angstroms (Å) and about 50 Angstroms generally pass through pore structures of the tumor tissue, but not that of normal tissue.

An elongated, worm-like conformation of a polymeric contrast agent results in greater uptake than other conformations, such as folded, or globular conformations. Conformation may be measured by a persistence length of the molecule. This may be determined by light scattering. When the polymeric backbone is in an elongated conformation, the chelator/MR active entity is free to rotate about its attachment point to the main chain, allowing a long T1 relaxation time of the surrounding water protons which are the source of the MR signal.

When the polymeric contrast agent is in a globular or highly folded conformation, steric hindrance, and molecular crowding causes interaction with the chelator/MR active entity restricting rotation about its bond to the main chain. Thus, the chelator/MR active entity moves only with the general slow motion of the carrier molecule. This produces a short T1 relaxation time.

A high relaxivity is associated with a molecule which folds upon itself into a globular conformation, such as albumen, at about 15 sec.−1 milliMolar−1 (sec.−1 mM−1). A low relaxivity is associated with an elongated molecule such as highly substituted Gd- DTPA polylysine in which the Gd can rotate rapidly, having a relaxivity of about 8 sec.−1 mM−1. The optimum conformation of the present invention is associated with a relaxivity of 7-8 sec.−1 mM−1. When the relaxivity of a peptide agent was high, the uptake coefficient of such an agent was invariably low, evidently due to the absence of the reptation mechanism.

Since many in-vivo chemical entities have a negative charge, molecules introduced into the subject can advantageously have a net negative charge to reduce agglutination and to allow for stable long circulation in the blood plasma. It is known that negatively charged dextran molecules undergo glomerular filtration at a much slower rate than equivalent dextran molecules of positive charge or neutral charge.

The following examples are included for purposes of illustrating certain aspects of the subject matter disclosed herein and should not be interpreted as limiting the scope of the overall disclosure herein.

### EXAMPLE 1

An animal model was used to demonstrate the MR imaging effects associated with angiogenesis. Fisher female rats were implanted subcutaneously with rat mammary adenocarcinoma cells (ATTC Mat B cells) that were grown to a suitable density in tissue culture. The implanted cells grew into tumors of 1-2 cm diameter in about 10 to 14 days and continued to grow to larger sizes when experiments extended beyond that time frame. The reptating polymer that was used (Polylysine-Gd-DTPA) was synthesized using a synthesis method described above with polymer length of 780 monomer units and molecular weight of 460kDaltons. The animals were injected intravenously at a dose of 0.025 mmoles Gd/kG. The imagining conditions include a T1 weighted spin echo pulse sequence (TR=250 ms, TE=9 ms), 12 centimeter FOV, 1 millimeter slices. The receive coil was a solenoid coil of about 5 centimeters diameter. Imaging was done on a Signa 1.5Tesla scanner.

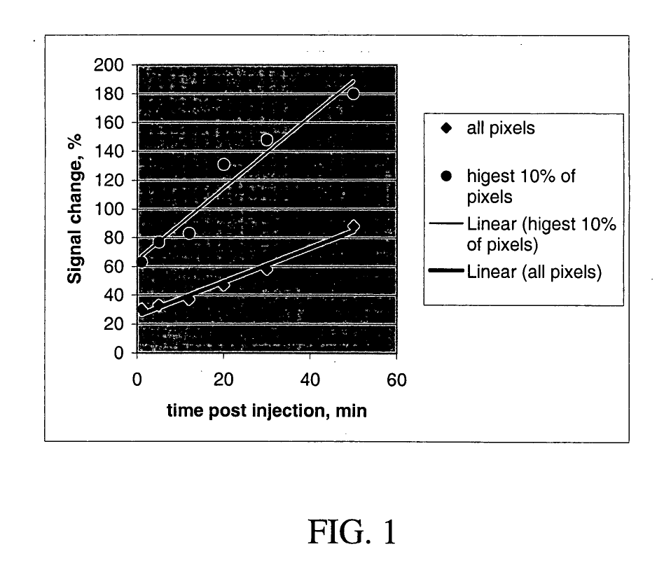

FIG. 1 is a graph of the signal slope determination with a series of images taken at intervals of 3 minutes for the first 10 minutes, then every 5 minutes up to 20 minutes, and finally every 10 minutes up to a final time of 60 minutes. The intercept is proportional to the vascular volume, and the slope gives the permeability of the tumor.

The signals are heterogenous in space due to angiogenesis being most prevalent in the outer rim of the tumor. Thus in order to capture the regions of interest with high permeability, the average signal associated with the pixels of the entire tumor in the image slice is plotted in addition to the average signal associated with the highest 10% of the pixels. The highest 10% of the pixels represents the hypermeable regions associated with angiogenic processes. The use of all pixels represents the average values for the entire tumor which includes then the center of tumors which are often necrotic and unreactive in terms of blood perfusion. These regions are not biologically relevant and thus the total average values have more “noise” that are associated with the necrotic processes which are not of direct interest in the present case.

While specific embodiments of the invention have been illustrated and described herein, it is realized that modifications and changes will occur to those skilled in the art. It is therefore to be understood that the appended claims are intended to cover all such modifications and changes as fall within the true spirit and scope of the invention.

What we claim is:

1. A method for measuring tumor response to a cancer treatment comprising the steps of:
a) injecting a polymeric contrast agent in a subject;
b) obtaining a series of magnetic resonance image signals of the contrast agent in the tumor for up to about 60 minutes; and
c) determining a slope of the magnetic resonance image signal as a function of time.

2. The method in accordance with claim 1, further comprising the step of:
d) determining the tumor signal at time zero.

3. The method in accordance with claim 1, further comprising:
e) treating a subject with a cancer treatment;
f) repeating steps (a) through (c) at least about 24 hours after the initial cancer treatment; and
g) comparing the first and second slopes in order to determine tumor response to the cancer treatment.

4. The method in accordance with claim 3, further comprising the step of:
h) determining the tumor signal at time zero; and
i) comparing the first and second tumor signals at time zero to determine tumor blood fractional volume.

5. The method in accordance with claim 1, wherein the step of obtaining a series of magnetic resonance image signals includes obtaining at least two magnetic resonance images.

6. The method in accordance with claim 1, wherein the step of obtaining a series of magnetic resonance image signals includes obtaining at least two magnetic resonance image signals in the first 10 minutes, obtaining at least one magnetic resonance image signals up to a total time of 20 minutes, and obtaining at least one magnetic resonance image signals up to a total time of 60 minutes.

7. The method in accordance with claim 1, wherein the step of injecting a polymeric contrast agent comprises injecting a dose in a range between about 0.01 mmoles of chelated imaging metal ion per kilogram of patient body weight and about 0.1 mmoles of chelated imaging metal ion per kilogram of patient body weight.

8. The method in accordance with claim 1, wherein the step of injecting a polymeric contrast agent comprising injecting a polypeptide containing lysine residues and optionally, one or more types of amino acid residues selected from the group consisting of glutamic acid residues and aspartic acid residues, wherein the lysine residues are substituted with a group derived from a steric hindrance molecule; and an image producing entity.

9. The method in accordance with claim 8, wherein the polypeptide has in a range between about 35 and about 1500 amino acid residues.

10. The method in accordance with claim 9, wherein the polypeptide has in a range between about 100 and about 800 amino acid residues.

11. The method in accordance with claim 8, wherein the image producing entity is a paramagnetic entity.

12. The method in accordance with claim 11, wherein the paramagnetic entity is gadolinium.

13. The molecule in accordance with claim 8, wherein the steric hindrance molecule is selected from the group consisting of diethylenetriaminepentaacetic acid (DTPA), 1,4,7,10-tetraazacyclododecane-1,4,7,10-tetraacetic acid (DOTA), 1,4,7,10-tetraazacyclododecane-1,4,7,10-tetrakis(2-propionic acid) (DOTMA), 1,4,8,11-tetraazacyclotetradecane-1,4,8,11-tetraacetic acid (TETA), 1,4,7,10-tetraazacyclododecane-1,4,7,10-tetrakis(3-(4-carboxyl)-butanoic acid), 1,4,7,10-tetraazacyclododecane-1,4,7,10-tetrakis(acetic acid-methyl amide), 1,4,7,10-tetraazacyclododecane-1,4,7,10-tetrakis(methylene phosphonic acid), and p-isothiocyanatobenzyl -1,4,7,10-tetraazacyclododecane-1,4,7,10-tetraacetic acid (p-SCN-Bz-DOTA).

14. The method in accordance with claim 13, wherein the steric hindrance molecule is 1,4,7,10-tetraazacyclododecane-1,4,7,10-tetraacetic acid (DOTA) or p-isothiocyanatobenzyl-1,4,7,10-tetraazacyclododecane-1,4,7,10-tetraacetic acid (p-SCN-Bz-DOTA).

15. The method in accordance with claim 8, wherein the polypeptide is a homopolymer of lysine.

16. The method in accordance with claim 8, wherein the substituted polypeptide has an average diameter in a range between about 20 angstroms and about 50 angstroms.

17. A method for measuring tumor response to a cancer therapy comprising the steps of:
a) injecting in a subject a homopolymer of lysine wherein lysine residues are substituted with Gd-DTPA;
b) obtaining a series of magnetic resonance image signals of the contrast agent in the tumor for up to about 60 minutes;
c) determining a slope of the magnetic resonance image signal as a function of time;
d) determining the tumor signal at time 0;
e) treating the subject with a cancer treatment;
f) repeating steps a) through d) at least 24 hours after the initial cancer treatment; and
g) comparing the first and second measurement to assess tumor response to the cancer therapy.
